(19) United States
(12) Patent Application Publication
(10) Pub. No.: US 2007/0185730 A1
(43) Pub. Date: Aug. 9, 2007

(54) Systems and methods for dynamic exam priority

(75) Inventors: Prakash Mahesh, Hoffman Estates, IL (US); Mark M. Morita, Arlington Heights, IL (US)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 11/348,163

(22) Filed: Feb. 6, 2006

(51) Int. Cl. G06Q 10/00; G05B 19/418

(52) U.S. Cl. 705/2; 705/8

(57) ABSTRACT

Certain embodiments of the present invention provide a system for exam prioritization including a priority indicator and a database. The priority indicator is assigned a priority level selected from at least three available priority levels. The at least three available priority levels represent categories of patient acuity. The database is adapted to store an association of the priority indicator and a medical exam. In certain embodiments, the priority indicator is adapted to by dynamically adjusted.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The present invention generally relates to medical workflow. In particular, the present invention relates to systems and methods for dynamic exam priority.

Healthcare environments, such as hospitals or clinics, include information management systems such as clinical information systems and storage systems. Clinical information systems may include, for example, hospital information systems (HIS) and radiology information systems (RIS). Storage systems may include, for example, picture archiving and communication systems (PACS). Information stored may include patient medical histories, imaging data, test results, diagnosis information, management information, and/or scheduling information, for example. The information may be centrally stored or divided at a plurality of locations. Healthcare practitioners may desire to access patient information or other information at various points in a healthcare workflow. For example, during surgery, medical personnel may access patient information, such as images of a patient's anatomy, that are stored in an information management system. Alternatively, medical personnel may enter new information, such as history, diagnostic, or treatment information, into an information management system during an ongoing medical procedure.

One example of an information management system is a PACS. PACS connect to medical diagnostic imaging devices and employ an acquisition gateway (between the acquisition device and the PACS), storage and archiving units, display workstations, databases, and sophisticated data processors. These components are integrated together by a communication network and data management system. A PACS has, in general, the overall goals of streamlining health-care operations, facilitating distributed remote examination and diagnosis, and improving patient care.

A typical application of a PACS system is to provide one or more medical images for examination by a medical professional. For example, a PACS system can provide a series of x-ray images to a display workstation where the images are displayed for a radiologist to perform a diagnostic examination. Based on the presentation of these images, the radiologist can provide a diagnosis. For example, the radiologist can diagnose a tumor or lesion in x-ray images of a patient's lungs.

A reading, such as a radiology or cardiology procedure reading, is a process of a healthcare practitioner, such as a radiologist or a cardiologist, viewing digital images of a patient. The practitioner performs a diagnosis based on the content of the diagnostic images and reports on results electronically (e.g., using dictation or otherwise) or on paper. The practitioner, such as a radiologist or cardiologist, typically uses other tools to perform diagnosis. Some examples of other tools are prior and related prior (historical) exams and their results, laboratory exams (such as blood work), allergies, pathology results, medication, alerts, document images, and other tools.

A clinical or healthcare environment is a crowded, demanding environment that would benefit from organization and improved ease of use of imaging systems, data storage systems, and other equipment used in the healthcare environment. A healthcare environment, such as a hospital or clinic, encompasses a large array of professionals, patients, and equipment. Personnel in a healthcare facility must manage a plurality of patients, systems, and tasks to provide quality service to patients. Healthcare personnel may encounter many difficulties or obstacles in their workflow.

With increasing volumes of examinations and images, a reduction of radiologists, and mounting pressures on improving productivity, radiologists and other healthcare personnel are in need of image processing or display workflow enhancements that aid in prioritizing workflow. Currently, healthcare personnel utilize worklists to organize and prioritize their workflow. Worklists show a list of exams or procedures, for example. A worklist may provide a list of exams for a radiologist to read, for example. Worklists may show new exams or procedures as new cases are created in the system. The worklist may allow the radiologist to organize the exams to be read based on time received or patient name, for example.

There is a need for workflow enhancements that allow a healthcare provider to attend to more acute cases first. Current systems only allow exams with higher priority to be marked “stat” as contrasted with exams having normal priority. That is, current systems support only a binary indication of priority for an exam. Within the group of exams marked as “stat,” there is no indication or organization of which cases have higher priority than others. Thus, there exists a need for a system and method for improved exam priority indication.

The decision to mark an exam as “stat” is typically made when the exam is ordered. Current systems do not permit medical personnel to change the priority after more information about the patient's condition has been obtained. That is, current systems do not allow an exam's priority to be changed dynamically from, for example, “stat” to “normal” or from “normal” to “stat.” For example, a technologist may be performing the exam originally assigned a normal priority. The technologist may determine that the patient is more critical than initially thought, but has no way to escalate the priority of the exam for reading by a radiologist, for example. Thus, there exists a need for a system and method for dynamic exam priority.

### BRIEF SUMMARY OF THE INVENTION

Certain embodiments of the present invention provide a system for exam prioritization including a priority indicator and a database. The priority indicator is assigned a priority level selected from at least three available priority levels. The at least three available priority levels represent categories of patient acuity. The database is adapted to store an association of the priority indicator and a medical exam.

Certain embodiments of the present invention provide a method for exam prioritization including assigning a priority indicator a priority level, associating the priority indicator and a medical exam, and storing the association of the priority indicator and the medical exam. The priority level is selected from at least three available priority levels. The at least three available priority levels represent categories of patient acuity.

Certain embodiments of the present invention provide a computer-readable medium including a set of instructions for execution on a computer, the set of instructions including a selection routine, an assignment routine, an association routine, and a storage routine. The selection routine is configured to allow a user to select a priority level from at least three available priority levels. The at least three available priority levels represent categories of patient acuity. The assignment routine is configured to assign the selected priority level to a priority indicator. The priority indicator is adapted to be dynamically adjusted. The association routine is configured to associate the priority indicator and a medical exam. The storage routine configured to store the association of the priority indicator and the exam.

### BRIEF DESCRIPTION OF SEVERAL VIEWS OF THE DRAWINGS

FIG. 1 illustrates an exemplary workflow for dynamic exam priority in accordance with an embodiment of the present invention.

FIG. 2 illustrates a system for exam prioritization in accordance with an embodiment of the present invention.

FIG. 3 illustrates a flow diagram for a method for dynamic exam priority in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

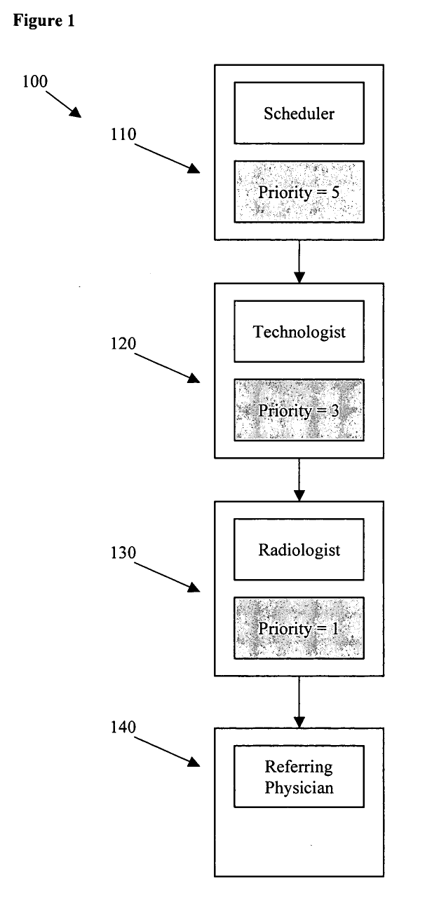

FIG. 1 illustrates an exemplary workflow 100 for dynamic exam priority in accordance with an embodiment of the present invention. The exemplary workflow 100 illustrated in FIG. 1 includes the following tasks, which will be described below in more detail. Task 110 includes entering an exam. Task 120 includes performing the exam. Task 130 includes reading the exam. Task 140 includes notification of a user.

Task 110 includes entering an exam. An exam may be entered and/or scheduled by a scheduler or other administrative personnel, for example. The exam may be entered into an information management system based on an order from a referring or admitting physician, for example. The exam may be associated with a priority indicator. The priority indicator may be assigned a priority level. For example, the priority indicator may be assigned the value of “5” indicating “normal” priority level. It should be noted that as illustrated in the embodiment in FIG. 1, a higher priority indicator value indicates a lower priority level. For example, a priority indicator value of “5” represents “normal” priority. In contrast, a priority indicator value of “1” represents “critical” priority. A “normal” priority level may be used for a routine exam, for example. As another example, the priority indicator may be assigned the value of “5” indicating “critical” priority level. A “critical” priority level may be used for a trauma case, where the patient was involved in a car accident, for example. Other priority levels may represent more or less patient acuity.

Task 120 includes performing an exam. The exam may be performed by a technologist or lab technician, for example. For example, a technologist may acquire images from an imaging modality.

A technologist and/or lab technician may utilize a worklist for determining what exams have been entered and/or scheduled. A plurality of exams, each having an associated priority indicator, may be ordered in the worklist. The exams may be ordered based on, for example, the associated priority indicators. The order an exam is performed in may be based at least in part on the priority indicator associated with the exam, for example.

In addition, while performing the exam, medical personnel, such as a technologist, may determine that a patient is more or less critical than previously thought. Thus, the technologist may alter the value of the priority indicator associated with the exam. For example, the technologist may determine that a priority indicator value of “3” is more appropriate to the patient's condition and may then alter the priority indicator value associated with the exam to reflect this, as illustrated in FIG. 1. A priority number of “3” may represent a “high” priority, for example.

Task 130 includes reading the exam. The exam may be read by a specialist, for example. For example, a radiologist or cardiologist may read an exam to make a diagnosis.

A specialist such as a radiologist may utilize a worklist for determining what exams are available to be read. A plurality of exams, each having an associated priority indicator, may be ordered in the worklist. The exams may be ordered based on, for example, the associated priority indicators. The order an exam is read may be based at least in part on the priority indicator associated with the exam, for example. For example, the exams in the worklist may be sorted based on the priority indicators associated with the exams, with exams associated with higher priority values ordered before exams associated with lower priority values.

In addition, while reading the exam, a healthcare practitioner, such as a radiologist, may determine that a patient is more or less critical than previously thought. Thus, the radiologist may alter the value of the priority indicator associated with the exam. For example, the radiologist may determine that a priority indicator value of “3” is more appropriate to the patient's condition and may then alter the priority indicator value associated with the exam to reflect this, as illustrated in FIG. 1. A priority number of “3” may represent a “high” priority, for example.

Task 140 includes notification of a user. Notification may include, for example, paging or sending an email. The notification may be made to one or more of a referring physician, treating physician, admitting physician, nurse, and/or technician, for example.

The notification to the referring physician may include an audit trail, for example. The referring physician may perform a follow-up on the case with greater urgency based on the notification, for example. That is, the referring physician may immediately follow-up with the patient, radiologist, and/or treating physician rather than waiting until a later time because of the notification. In an embodiment, the referring physician's review of the notification may trigger a notification to the radiologist informing him of the referring physician's review of the now higher priority case.

In certain embodiments, a change in the priority indicator associated with the exam may trigger a notification. The notification may be initiated because the priority indicator associated with an exam exceeded a threshold value, for example. For example, a notification may be initiated when the priority indicator associated with an exam is adjusted to a value greater than “4,” indicating “very high” priority. As another example, the notification may be initiated when the value of the priority indicator is adjusted by a particular amount. For example, the notification may be initiated because the priority indicator has cone up two values. As another example, the notification may be initiated when the priority indicator is adjusted more than one time. For example, multiple adjustments in the value of the priority indicator may indicate a worsening of the patient's condition, triggering a notification.

Certain embodiments of the present invention may omit one or more of the tasks in exemplary workflow 100 and/or perform the tasks in a different order than the order listed. For example, some tasks may not be performed in certain embodiments of the present invention. As a further example, certain tasks may be performed in a different temporal order, including simultaneously, than listed above.

One or more of the tasks of the exemplary workflow 100 may be implemented alone or in combination, in part or in whole, in hardware, firmware, and/or as a set of instructions in software, for example. Certain embodiments may be provided as a set of instructions residing on a computer-readable medium, such as a memory or hard disk, for execution on a general purpose computer or other processing device, such as, for example, a PACS workstation or image viewer.

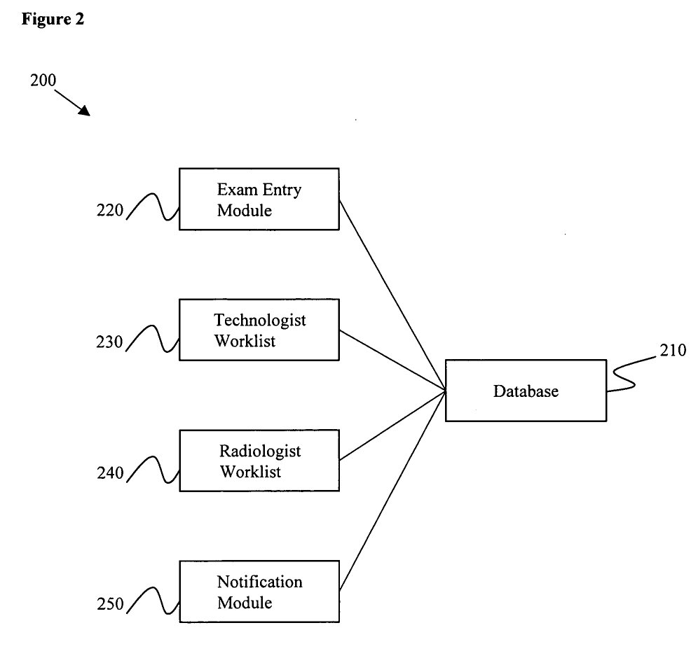

FIG. 2 illustrates a system 200 for exam prioritization in accordance with an embodiment of the present invention. The system 200 includes a database 210, an exam entry module 220, a technologist worklist 230, a radiologist worklist 240, and a notification module 250.

The database 210 is in communication with the exam entry module 220, the technologist worklist 230, the radiologist worklist 240, and the notification module 250.

In operation, an exam is entered and/or scheduled. The exam may be entered and/or scheduled in an information management system. The exam may be entered into an HIS based on an order from a referring or admitting physician, for example. The exam may be entered by exam entry module 220, for example. The exam entry module 220 may be, for example, an order entry application. As another example, the exam entry module 220 may be a scheduling application. The exam entry module 220 may be used by a scheduler or other administrative personnel, for example. In an embodiment, the exam entry module 220 is adapted to allow a user to enter and/or schedule an exam or procedure.

In an embodiment, the exam entry module 220 is adapted to allow a priority level to be selected. In an embodiment, the priority level may be selected at least in part by a user. The priority level may be selected by a user using the exam entry module 220, for example. The priority level may be selected from a set of available priority levels. For example, the available priority levels may include “normal,” “low,” “high,” “elevated,” “very high,” and/or “critical.” As another example, the priority levels may include “normal” and “stat.” In certain embodiments, available priority levels may use a variety of names, conventions, numbers, and/or ranges and that many other available priority levels may be used in addition to the examples just given. Different priority levels may indicate different degrees, levels, and/or categorizations of patient acuity, for example. A “normal” priority level may be used for a routine exam, for example. As another example, the priority indicator may be assigned the value of “5” indicating “critical” priority level. A “critical” priority level may be used for a trauma case, where the patient was involved in a car accident, for example.

In an embodiment, one or more of the available priority levels are configured for a specific healthcare facility or environment, such as a hospital, clinic, or network of providers. For example, an oncology clinic may assign different or more fine-grained priority values to a broader or more detailed list of diagnoses, conditions, anomalies, and/or abnormalities than a general care facility.

In an embodiment, the selected priority level is assigned to a priority indicator. In an embodiment, the priority indicator is associated with the exam. The association between the exam and the priority indicator may be stored in database 210. The database 210 may be part of an information management system, for example. For example, the database 210 may be part of a PACS or RIS. In an embodiment, the database 210 may be implemented as a table, interpreted code, database query, or other data structure. In an embodiment, the priority indicator may be dynamically adjusted. That is, the value of the priority indicator may be changed after it has been set to a value.

The technologist worklist 230 allows a user to view, organize, and/or process worklist entries. The technologist worklist 230 may be used by a technologist or lab technician, for example. A worklist entry may correspond to a particular exam, procedure, and/or study, for example. A technologist and/or lab technician may use the technologist worklist 230 to determine, for example, in what order and/or what modality should be used for a given exam and/or procedure.

In certain embodiments, the technologist worklist 230 may retrieve the priority indicator for an exam from the database 210. In certain embodiments, the technologist worklist 230 may retrieve the association of an exam and a priority indicator from the database 210. The technologist worklist 230 may provide various mechanisms to order worklist entries such as exams and/or procedures. For example, a lab technician may utilize the technologist worklist 230 to order a set of scheduled exams. For example, a user may select a button to display recently scheduled exams. As another example, the entries in the technologist worklist 230 may be sorted based on some criteria. For example, a technologist may utilize the technologist worklist 230 to sort exams based at least in part on the priority indicator associated with those exams. For example, technologist worklist 230 may display worklist entries from highest to lowest priority level. Thus, exams with higher priority indicator values may be listed before exams having lower priority indicator values in the technologist worklist 230. In an embodiment, the technologist worklist 230 may color-code worklist entries based at least in part on the priority indicator associated with an exam. In an embodiment, technologist worklist 230 is automatically sorted based on the priority level. In an embodiment, within a set of exams with the same priority level, the time a particular exam was scheduled may also be used to further organize the technologist worklist 230.

In an embodiment, the technologist worklist 230 is capable of dynamically adjusting the value of the priority indicator. In an embodiment, the technologist worklist 230 allows a user to dynamically adjust the value of the priority indicator. That is, the technologist worklist 230 allows a user to change the value of the priority indicator after it has been set. For example, a technologist may be performing an exam originally assigned a “normal” priority. The technologist may determine that the patient is more critical than initially thought, and may adjust the priority indicator associated with the exam to have a value of “high” priority. In an embodiment, the technologist worklist 230 is adapted to store the adjusted priority indicator in the database 210.

The radiologist worklist 240 may be similar to the technologist worklist 230, described above, for example. The radiologist worklist 240 allows a user to view, organize, and/or process worklist entries. The radiologist worklist 240 may be used by a radiologist, cardiologist, or other specialist, for example. A worklist entry may correspond to a particular exam, procedure, and/or study, for example. A radiologist may use the radiologist worklist 240 to determine, for example, in what order to read a particular exam and/or study.

In certain embodiments, the radiologist worklist 240 may retrieve the priority indicator for an exam from the database 210. In certain embodiments, the radiologist worklist 240 may retrieve the association of an exam and a priority indicator from the database 210. The radiologist worklist 240 may provide various mechanisms to order worklist entries such as exams and/or procedures. For example, a radiologist may utilize the radiologist worklist 240 to order a set of exams to be read. For example, a user may select a button to display recently performed exams. As another example, the entries in the radiologist worklist 240 may be sorted based on some criteria. For example, a radiologist may utilize the radiologist worklist 240 to sort exams based at least in part on the priority indicator associated with those exams. For example, radiologist worklist 240 may display worklist entries from highest to lowest priority level. Thus, exams with higher priority indicator values may be listed before exams having lower priority indicator values in the radiologist worklist 240. In an embodiment, the radiologist worklist 240 may color-code worklist entries based at least in part on the priority indicator associated with an exam. In an embodiment, radiologist worklist 240 is automatically sorted based on the priority level. In an embodiment, within a set of exams with the same priority level, the time a particular exam was performed may also be used to further organize the radiologist worklist 240.

In an embodiment, the radiologist worklist 240 is capable of dynamically adjusting the value of the priority indicator. In an embodiment, the radiologist worklist 240 allows a user to dynamically adjust the value of the priority indicator. That is, the radiologist worklist 240 allows a user to change the value of the priority indicator after it has been set. For example, a radiologist may be reading an exam originally assigned a “normal” priority. The radiologist may determine that the patient is more critical than initially thought, and may adjust the priority indicator associated with the exam to have a value of “stat” priority. In an embodiment, the radiologist worklist 240 is adapted to store the adjusted priority indicator in the database 210.

In certain embodiments, the system 200 may include a notification module 250. The notification module 250 is capable of initiating a notification. The notification module 250 is capable of notifying one or more recipients. A recipient may be, for example, a referring physician, a treating physician, and/or a nurse. As another example, a recipient may be a software module or program. As another example, a recipient may be a component and/or device such as a notification inbox or message manager. A notification inbox may allow a user to organize and/or respond to received notifications, for example.

A recipient may be notified by one or more media. For example, a recipient may be paged and/or emailed. The notification to the recipient may include information about the patient, exam, and/or priority level, for example. For example, the notification may include a patient's name, the exam to be performed, an audit trail, and information about what event or events caused the notification to be initiated.

In certain embodiments, a change in the priority indicator associated with the exam may trigger a notification. The notification may be initiated because the priority indicator associated with an exam exceeded a threshold value, for example. For example, a notification may be initiated when the priority indicator associated with an exam is adjusted to a value greater than “4,” indicating “very high” priority. As another example, the notification may be initiated when the value of the priority indicator is adjusted by a particular amount. For example, the notification may be initiated because the priority indicator has cone up two values. As another example, the notification may be initiated when the priority indicator is adjusted more than one time. For example, multiple adjustments in the value of the priority indicator may indicate a worsening of the patient's condition, triggering a notification.

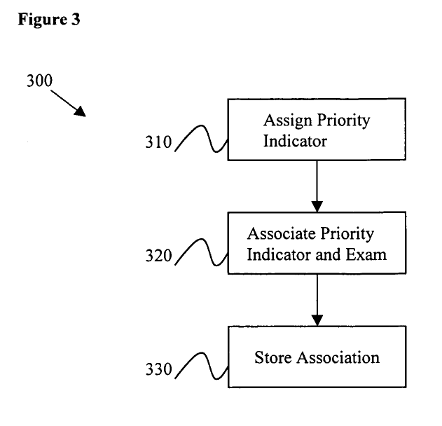

FIG. 3 illustrates a flow diagram for a method 300 for dynamic exam priority in accordance with an embodiment of the present invention. The method 300 includes the following steps, which will be described below in more detail. At step 310, a priority indicator is assigned. At step 320, a priority indicator is associated with an exam. At step 330, an association is stored. The method 300 is described with reference to elements of systems described above, but it should be understood that other implementations are possible.

At step 310, a priority indicator is assigned. The priority indicator may be assigned a priority level, for example. The priority indicator may indicate the priority level of an exam, for example. The priority level may be selected by an exam entry module, similar to exam entry module 220, described above, for example. In an embodiment, the priority level may be selected at least in part by a user. The priority level may be selected by a user using the exam entry module 220, for example.

The priority level may be selected from a set of available priority levels. For example, the available priority levels may include “normal,” “low,” “high,” “elevated,” “very high,” and/or “critical.” As another example, the priority levels may include “normal” and “stat.” In an embodiment, one or more of the available priority levels are configured for a specific healthcare facility or environment, such as a hospital, clinic, or network of providers. For example, an oncology clinic may assign different or more fine-grained priority values to a broader or more detailed list of diagnoses, conditions, anomalies, and/or abnormalities than a general care facility.

At step 320, a priority indicator is associated with an exam. The priority indicator may be the priority indicator assigned in step 310, described above, for example. The exam may be entered and/or scheduled by a scheduler or other administrative personnel, for example. The exam may be entered and/or scheduled in an information management system. The exam may be entered into an HIS based on an order from a referring or admitting physician, for example. The exam may be entered by exam entry module, similar to exam entry module 220, described above, for example. The exam entry module 220 may be, for example, an order entry application. As another example, the exam entry module 220 may be a scheduling application. The exam entry module 220 may be used by a scheduler or other administrative personnel, for example. In an embodiment, the exam entry module 220 is adapted to allow a user to enter and/or schedule an exam or procedure.

At step 330, an association is stored. The association may be the association between the priority indicator and the exam, described above at step 320, for example. The association between the exam and the priority indicator may be stored in database similar to database 210, described above, for example. The database 210 may be part of an information management system, for example. For example, the database 210 may be part of a PACS or RIS. In an embodiment, the database 210 may be implemented as a table, interpreted code, database query, or other data structure.

In an embodiment, a medical exam may be processed based at least in part on the associated priority indicator. For example, a worklist may order a plurality of exams based at least in part on their priority. The worklist may be similar to the technologist worklist 230 and/or the radiologist worklist 240, both described above, for example. As another example, processing, retrieval, manipulation, and/or storage of the exam may be based on the priority level.

In an embodiment, the priority indicator may be adjusted. The priority indicator may be dynamically adjusted. That is, the value of the priority indicator may be changed after it has been set. The priority indicator may be adjusted by a worklist, similar to the technologist worklist 230 and/or the radiologist worklist 240, both described above, for example. For example, a technologist may be performing an exam originally assigned a “normal” priority. The technologist may determine that the patient is more critical than initially thought, and may adjust the priority indicator associated with the exam to have a value of “high” priority. In an embodiment, the priority indicator may be adjusted based at least in part on input from a user.

In an embodiment, a notification is initiated. The notification may be initiated by a notification module. The notification module may be similar to notification module 250, described above, for example. The notification may be sent to one or more recipients. A recipient may be, for example, a referring physician, a treating physician, and/or a nurse. As another example, a recipient may be a software module or program. As another example, a recipient may be a component and/or device such as a notification inbox or message manager. A notification inbox may allow a user to organize and/or respond to received notifications, for example.

A recipient may be notified by one or more media. For example, a recipient may be paged and/or emailed. The notification to the recipient may include information about the patient, exam, and/or priority level, for example. For example, the notification may include a patient's name, the exam to be performed, an audit trail, and information about what event or events caused the notification to be initiated.

In certain embodiments, the notification may be initiated based at least in part on a change in the priority indicator associated with an exam. The notification may be initiated because the priority indicator associated with an exam exceeded a threshold value, for example. For example, a notification may be initiated when the priority indicator associated with an exam is adjusted to a value greater than “4,” indicating “very high” priority. As another example, the notification may be initiated when the value of the priority indicator is adjusted by a particular amount. For example, the notification may be initiated because the priority indicator has cone up two values. As another example, the notification may be initiated when the priority indicator is adjusted more than one time. For example, multiple adjustments in the value of the priority indicator may indicate a worsening of the patient's condition, triggering a notification.

Certain embodiments of the present invention may omit one or more of these steps and/or perform the steps in a different order than the order listed. For example, some steps may not be performed in certain embodiments of the present invention. As a further example, certain steps may be performed in a different temporal order, including simultaneously, than listed above.

Thus, certain embodiments of the present invention provide a system and method for improved exam priority indication. In addition, certain embodiments of the present invention provide a system and method for dynamic exam priority. Certain embodiments provide a technical effect of improved exam priority indication, for example. Certain embodiments provide a technical effect of dynamic exam priority, for example.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A system for exam prioritization, the system including:
a priority indicator, wherein the priority indicator is assigned a priority level selected from at least three available priority levels, wherein the at least three available priority levels represent categories of patient acuity; and
a database, wherein the database is adapted to store an association of the priority indicator and a medical exam.

2. The system of claim 1, wherein the priority indicator is adapted to be dynamically adjusted.

3. The system of claim 1, wherein the at least one of the at least three available priority levels is customized for a specific healthcare environment.

4. The system of claim 1, further including an exam entry module, wherein the exam entry module is adapted to allow the medical exam to be entered into an information management system.

5. The system of claim 4, wherein the exam entry module is adapted to allow selection of the priority level.

6. The system of claim 1, further including a worklist, wherein the worklist is adapted to order a plurality of exams, wherein the plurality of exams includes the medical exam, wherein each exam in the plurality of exams is associated with a priority indicator, wherein the worklist is adapted to order the exams based at least in part on the priority indicators associated with the exams.

7. The system of claim 1, further including a worklist, wherein the worklist is adapted to allow the priority indicator to be adjusted.

8. The system of claim 7, wherein the priority indicator is adjusted based at least in part on input from a user.

9. The system of claim 1, further including a notification module, wherein the notification module is adapted to initiate a notification, wherein the notification is initiated based at least in part on the priority indicator.

10. The system of claim 9, wherein the notification is initiated based at least in part on a change in the priority indicator.

11. A method for exam processing, the method including:
assigning a priority indicator a priority level selected from at least three available priority levels, wherein the at least three available priority levels represent categories of patient acuity;
associating the priority indicator and a medical exam; and
storing the association of the priority indicator and the medical exam.

12. The method of claim 11, further including processing the medical exam based at least in part on the associated priority indicator.

13. The method of claim 12, wherein the processing step includes ordering a plurality of exams, wherein the plurality of exams includes the medical exam, wherein each exam in the plurality of exams is associated with a priority indicator, wherein the order of the exams is based at least in part on the priority indicators associated with the exams.

14. The method of claim 11, further including adjusting the priority indicator.

15. The method of claim 14, wherein the priority indicator is adjusted based at least in part on input from a user.

16. The method of claim 11, further including initiating a notification, wherein the notification is initiated based at least in part on the priority indicator.

17. The system of claim 16, wherein the notification is initiated based at least in part on a change in the priority indicator.

18. A computer-readable medium including a set of instructions for execution on a computer, the set of instructions including:
a selection routine configured to allow a user to select a priority level from at least three available priority levels, wherein the at least three available priority levels represent categories of patient acuity;
an assignment routine configured to assign the selected priority level to a priority indicator, wherein the priority indicator is adapted to be dynamically adjusted;
an association routine configured to associate the priority indicator and a medical exam; and
a storage routine configured to store the association of the priority indicator and the exam.

19. The set of instructions of claim 18, further including a processing routine configured to process the exam based at least in part on the associated priority indicator.

20. The set of instructions of claim 18, further including a notification routine configured to initiate a notification based at least in part on the priority indicator.
